(19) United States
(12) Patent Application Publication
(10) Pub. No.: US 2011/0264220 A1
(43) Pub. Date: Oct. 27, 2011

(54) DEVICE AND METHOD FOR DELIVERING RADIATION

(75) Inventor: Keith E. Miller, Germantown, TN (US)

(73) Assignee: WARSAW ORTHOPEDIC, INC., Warsaw, IN (US)

(21) Appl. No.: 12/766,541

(22) Filed: Apr. 23, 2010

(51) Int. Cl. A61F 2/44; A61N 5/00

(52) U.S. Cl. 623/17.16; 600/3

(57) ABSTRACT

Embodiments of the invention include a device for supplementing or replacing a spinal structure and therapeutically delivering radiation to tissue. Some embodiments include a plug with an integrated radiation source. The plug may be coupled to an interbody spinal implant or a vertebral body replacement implant.

### FIELD OF THE INVENTION

The present invention relates generally to the field of supplementing or replacing orthopedic structures, and more particularly relates to supplementing or replacing orthopedic structures with an implant and attaching a radiation source to the implant to treat tissue near the implant when in place in a patient's body.

### BACKGROUND

In some circumstances, an implant is used to supplement or replace an orthopedic structure. Such an implant may be used to respond to a spinal pathology, as part of a cancer treatment, or for any effective purpose or combination of purposes. For example and without limitation, an implant may be an interbody spinal implant or a vertebral body replacement implant. Implants classified as vertebral body replacement implants may include implants used in association with corpectomy or vertebrectomy procedures to stabilize spinal structures. Removal, or excision, of a vertebra may be referred to as a vertebrectomy. Excision of a generally anterior portion, or vertebral body, of the vertebra may be referred to as a corpectomy. If only a portion of a vertebral body and adjacent discs are removed and replaced, the procedure may be called a hemi-vertebrectomy. In accordance with the prior art, the implant used may also serve as a platform to assist with the delivery of radiation treatment toward adjacent tissues suspected of including or known to include one or more of cancerous cells and tumors. An improved radiation source may include integration with an implant. Some improved devices may be easily coupled with the implant to apply radiation treatment in a prescribed direction. Some improved devices may be capable of integration with an implant to direct treatment in one or more specified directions, and some may include mechanisms for controlling the intensity of the treatment applied. An improved device may be configured to couple with existing implants such as one or more of vertebral body replacement implants and interbody implants.

### SUMMARY

One embodiment of the invention is a device for supplementing or replacing a spinal structure and therapeutically delivering radiation to tissue within or near the spinal structure. The device may include an interbody spinal implant or a vertebral body replacement implant configured to be placed between a first vertebra and a second vertebra to supplement or replace at least a portion of the spinal structure. The interbody spinal implant or vertebral body replacement implant may include at least one opening. The device may also have a plug coupled to the interbody spinal implant or vertebral body replacement implant by at least in part occupying some portion of the at least one opening, and a radiation source integrated with the plug and configured to deliver radiation.

An embodiment of the invention is a plug configured to couple with an interbody spinal implant or a vertebral body replacement implant by at least in part occupying some portion of an opening in the interbody spinal implant or the vertebral body replacement implant. The plug may include a body having a first end and a substantially opposite second end and having at least some open space within the body. The plug may be made at least in part from a material that substantially blocks the transmission of radiation to direct radiation from only one or both of the first end and the second end of the plug.

Another embodiment of the invention is a method of irradiating cells near an interbody spinal implant or a vertebral body replacement implant. The method may include providing a plug configured to couple with the interbody spinal implant or the vertebral body replacement implant by at least in part occupying some portion of an opening in the interbody spinal implant or the vertebral body replacement implant. The plug may also include a radiation source. Some method embodiments include coupling the plug to the interbody spinal implant or the vertebral body replacement implant, inserting the interbody spinal implant or the vertebral body replacement implant into a patient, and inserting the plug into a patient such that radiation is directed toward a therapeutically effective location near the interbody spinal implant or the vertebral body replacement implant

### BRIEF DESCRIPTION OF THE DRAWINGS

FIG. 1 is a perspective view with a partial cut-away of an embodiment of a device for supplementing orthopedic structures and therapeutically delivering radiation to tissue.

FIG. 2 is a perspective view of an embodiment of a device for supplementing orthopedic structures and therapeutically delivering radiation to tissue.

FIG. 3 is a perspective view of an embodiment of a plug.

FIG. 4 is a perspective view of an embodiment of a plug.

FIG. 5 is a cross-sectional view of the plug of FIG. 3.

FIG. 6 is a cross-sectional view of the plug of FIG. 4.

FIG. 7 is a cross-sectional view of an embodiment of a plug.

FIG. 8 is a cross-sectional view of an embodiment of a plug.

### DETAILED DESCRIPTION

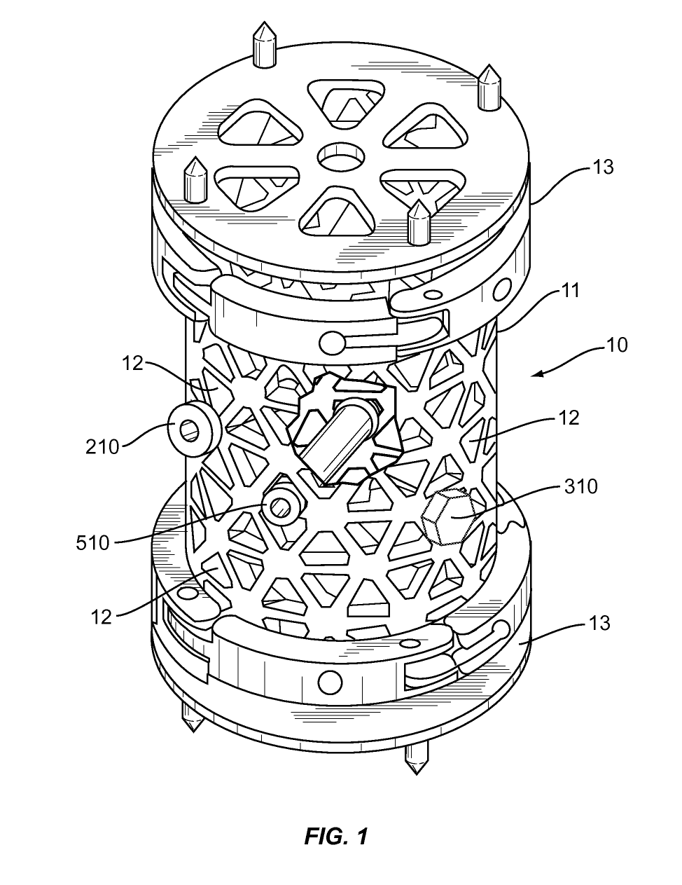

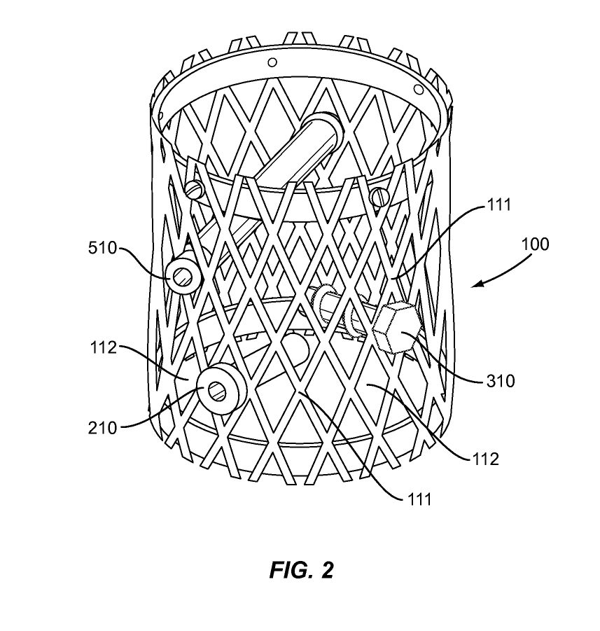

Embodiments of a device for supplementing or replacing spinal structures and therapeutically delivering radiation to tissue within or near the spinal structures are illustrated in FIGS. 1 and 2. In FIG. 1, a vertebral body replacement implant 10 in the form of a PYRAMESH® Surgical Titanium Mesh 11 is illustrated with a pair of SCEPTOR™ Universal Endcleats 13, all available from Medtronic, Inc. The vertebral body replacement implant 10 includes generally polygonal shaped openings 12 that are generally triangular.

In FIG. 2, a vertebral body replacement implant 110 is shown. The vertebral body replacement implant 110 includes a titanium mesh body 111 with generally four-sided openings 112 having approximately diamond shapes. The vertebral body replacement implants 10, 110 may be implants used in association with corpectomy, vertebrectomy, hemi-vertebrectomy, or any other spinal procedure that includes supplementing or replacing a spinal structure.

The openings of any embodiment of a vertebral body replacement implant or an embodiment of an interbody spinal implant may include holes, slots, indentations, or mechanisms of any functional shape. The vertebral body replacement implants 10, 110 illustrated have a generally round lateral periphery, but other embodiments may be shaped in any effective manner. A spinal interbody implant of embodiments of the device may be any implant that may be used in an interbody space between two vertebrae and may include at least on opening. Any embodiment of a spinal interbody implant or a vertebral body replacement implant of embodiments of the invention may supplement or replace at least a portion of a spinal structure treated. The lateral periphery of an interbody spinal implant or a vertebral body replacement implant may be substantially an oval, kidney shape, triangle, rectangle, square, any polygonal or curved shape, or any combination of shapes. In addition or as an alternative to titanium, the implants may be made from any biocompatible material. For example and without limitation, the mesh or other material of the implants may be made in whole or in part from polyetheretherketone (PEEK) or a PEEK composite, cobalt chrome, stainless steel, and any biocompatible metal, metal alloy, or polymer. The implants may also include a bone or bone-based material. For example and without limitation, the implants may include in whole or in part one or more of allograft, xenograft, demineralized bone, and autograft. In some embodiments, the vertebral body replacement implants may be configured to expand from a first height of a second taller height.

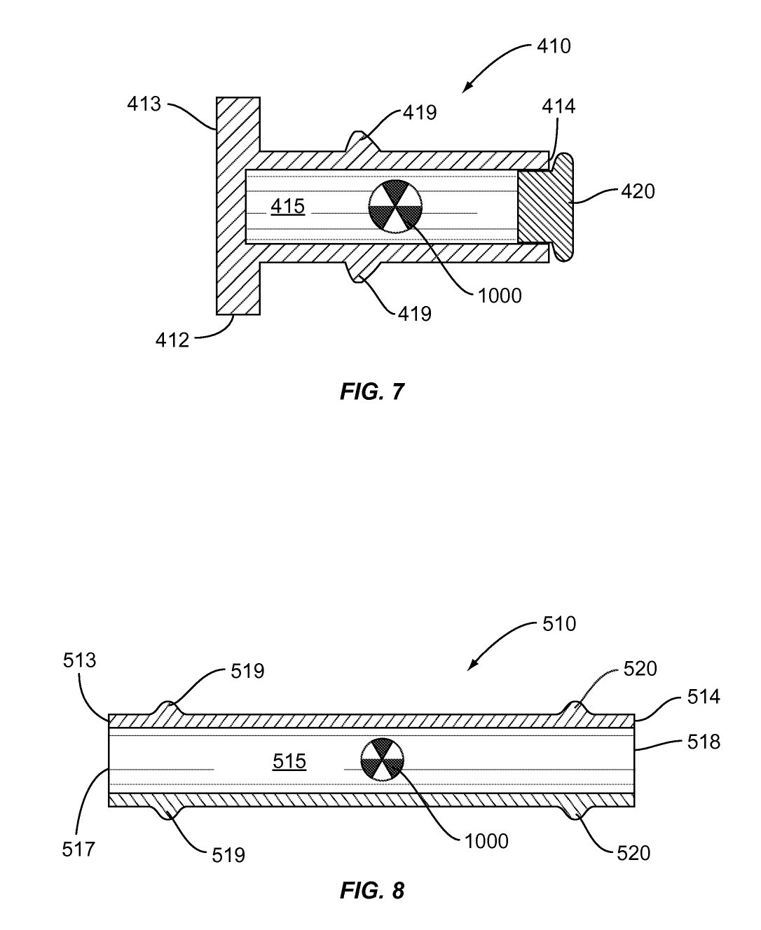

Embodiments of the device for supplementing or replacing a spinal structure may include a plug coupled to the interbody spinal implant or vertebral body replacement implant by at least in part occupying some portion of an opening in the implant. For example, a plug 210 is illustrated in FIGS. 1-3 and 5. Alternative or additional plugs are also illustrated that may be part of the device. Particularly, a plug 310 is shown in FIGS. 1, 2, 4, and 6. A plug 410 is illustrated in FIG. 7, and a plug 510 is shown in FIGS. 1, 2, and 8.

The plug 210 is coupled to the vertebral body replacement implant 10 (FIG. 1) by at least in part occupying a portion of the opening 12. The plug 210 is coupled to the vertebral body replacement implant 110 (FIG. 2) by at least in part occupying a portion of the opening 112. In other embodiments, a less significant portion of the plug 210 may occupy an opening in one of the implants. For example and without limitation, a plug may be primarily attached to an outer surface of an implant and include a minor portion that enters an opening in an implant.

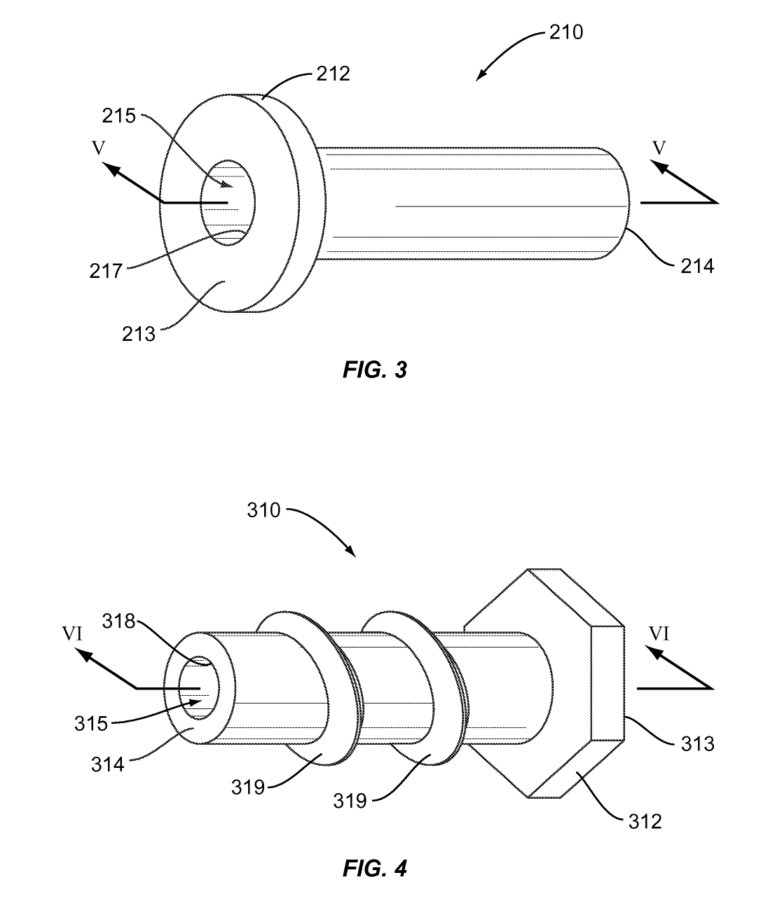

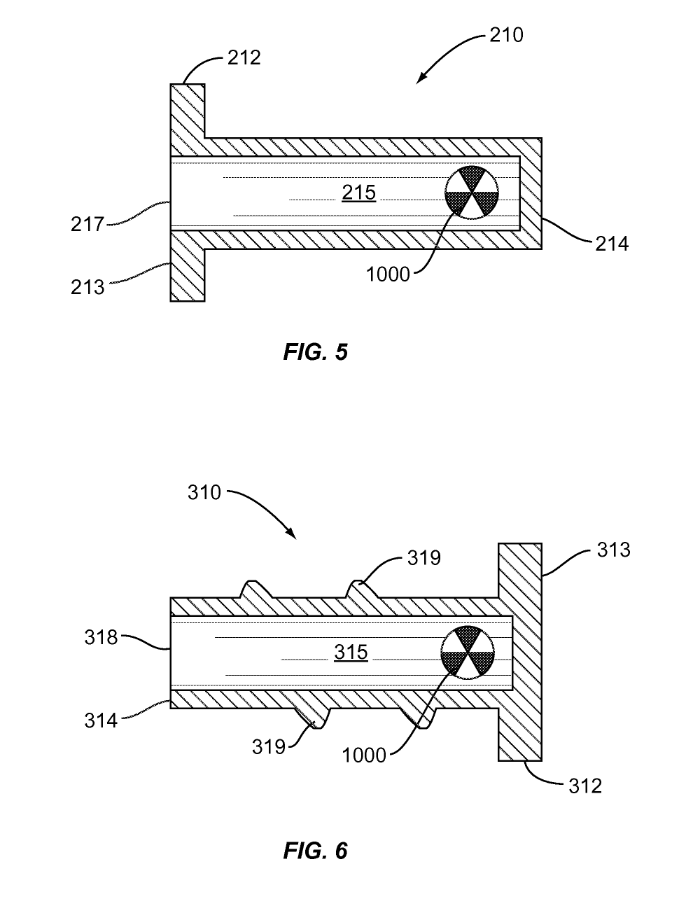

The plug 210 includes a flange 212, as illustrated in FIGS. 3 and 5, at a first end 213 of the plug 210. The flange 212 may provide a stop to limit the travel of the plug 210 placed into an opening, such as the openings 12, 112 (FIGS. 1 and 2) in a vertebral body replacement implant. The flange 212 may also provide an area by which the plug 210 may be pushed into or pulled from an opening or otherwise grasped. A connection mechanism of some embodiments may include an external hex, an internal hex, various flat portions, holes for receiving other components of a torque or other force delivering tool, or any other effective mechanism to connect with and manipulate the plug 210 from any end or side. In some embodiments, a connection mechanism is separate and distinct from a flange. The plug 210 shown includes a body extending between the first end 213 and a substantially opposite end 214. The plug 210 includes an open space 215 within the plug body. In other embodiments, for example where a radiation source is integrated into the material of a plug, the plug may be solid and not include an open space in the body. A plug designated a solid herein may be composed of more than one solid material and still be considered a solid. That is, designation as a solid does not necessarily mean that a plug is a homogeneous material.

The plug 210 includes a hole 217 from the open space 215 through the first end 213. In some embodiments, multiple holes may be provided from the open space 215 through the first end 213. In the embodiment shown, the second end 214 of the body is closed. This closure may be by a radiolucent material in some embodiments where it is desirable for radiation to emanate from the second end, or the closure may be made with a material that substantially blocks the transmission of radiation. In other embodiments, the body of a plug may include a hole from an open space through a second end and include a closed first end. In still other embodiments, the body of a plug may include holes from an open space through both the first and second ends. In some embodiments, no holes from an open space are provided.

The plug 310 is illustrated in FIGS. 1, 2, 4, and 6. The plug 310 is coupled to the vertebral body replacement implant 10 (FIG. 1) by at least in part occupying a portion of the opening 12. The plug 310 is coupled to the vertebral body replacement implant 110 (FIG. 2) by at least in part occupying a portion of the opening 112. In other embodiments, a less significant portion of the plug 310 may occupy an opening in one of the implants. For example and without limitation, a plug may be primarily attached to an outer surface of an implant and include a minor portion that enters an opening in an implant.

The plug 310 includes a flange 312, as illustrated in FIGS. 4 and 6, at a first end 313 of the plug 310. The flange 312 may provide a stop to limit the travel of the plug 310 placed into an opening, such as the openings 12, 112 (FIGS. 1 and 2) in a vertebral body replacement implant. The flange 312 may also provide an area by which the plug 310 may be pushed into or pulled from an opening or otherwise grasped. The flange 312 shown includes a connection mechanism in the form of an external hex fitting for applying torque to the plug 310. In other embodiments, a connection mechanism may include an internal hex, various flat portions, holes for receiving other components of a torque or other force delivering tool, or any other effective mechanism to connect with and manipulate the plug 310 from any end or side. In some embodiments, a connection mechanism is separate and distinct from a flange. The plug 310 shown includes a body extending between the first end 313 and a substantially opposite end 314. The plug 310 includes an open space 315 within the plug body. In other embodiments, for example where a radiation source is integrated into the material of a plug, the plug may be solid and not include an open space in the body. A plug designated a solid herein may be composed of more than one solid material and still be considered a solid. That is, designation as a solid does not necessarily mean that a plug is a homogeneous material.

The plug 310 illustrated includes a hole 318 from the open space 315 through the second end 314. In some embodiments, multiple holes may be provided from the open space 315 through the second end 314. In the embodiment shown, the first end 313 of the body is closed. This closure may be by a radiolucent material in some embodiments where it is desirable for radiation to emanate from the first end, or the closure may be made with a material that substantially blocks the transmission of radiation. In other embodiments, the body of a plug may include a hole from an open space through a first end and include a closed second end. In still other embodiments, the body of a plug may include holes from an open space through both the first and second ends. In some embodiments, no holes from an open space are provided.

The plug 310 includes threads 319 along at least a portion of the plug 310. The threads 319 of some embodiments are for engaging with an interbody spinal implant or a vertebral body replacement implant, such as the vertebral body replacement implants 10, 110 illustrated in FIGS. 1 and 2 respectively. The threads 319 may also be classified as protrusions along the plug 310 that engage with an interbody spinal implant or a vertebral body replacement implant. The threads 319 may serve to both advance the plug 310 into and keep the plug 310 from sliding or otherwise translating relative to an interbody spinal implant or a vertebral body replacement implant.

The plug 410 is similar to the plug 310, but includes protrusions 419 rather than threads. The plug 410 is illustrated in FIG. 7, and may be coupled to an interbody implant or a vertebral body replacement implant by at least in part occupying a portion of an opening in an implant. The plug 410 includes a flange 412 at a first end 413 of the plug 410. The flange 412 may provide a stop to limit the travel of the plug 410 placed into an opening, such as the openings 12, 112 (FIGS. 1 and 2) in a vertebral body replacement implant. The flange 412 may also provide an area by which the plug 410 may be pushed into or pulled from an opening or otherwise grasped. The flange 412 may be round, or any effective shape, or include a connection mechanism in the form of an external hex fitting for applying torque to the plug 410. In other embodiments, a connection mechanism may include an internal hex, various flat portions, holes for receiving other components of a torque or other force delivering tool, or any other effective mechanism to connect with and manipulate the plug 410 from any end or side. In some embodiments, a connection mechanism is separate and distinct from a flange. The plug 410 shown includes a body extending between the first end 413 and a substantially opposite end 414. The plug 410 includes an open space 415 within the plug body. In other embodiments, for example where a radiation source is integrated into the material of a plug, the plug may be solid and not include an open space in the body. A plug designated a solid herein may be composed of more than one solid material and still be considered a solid. That is, designation as a solid does not necessarily mean that a plug is a homogeneous material.

The plug 410 includes a hole 418 from the open space 415 through the second end 414. In some embodiments, multiple holes may be provided from the open space 415 through the second end 414. In the embodiment shown, the first end 413 of the body is closed. This closure may be by a radiolucent material in some embodiments where it is desirable for radiation to emanate from the first end, or the closure may be made with a material that substantially blocks the transmission of radiation. In other embodiments, the body of a plug may include a hole from an open space through a first end and include a closed second end. In still other embodiments, the body of a plug may include holes from an open space through both the first and second ends. In some embodiments, no holes from an open space are provided. A stopper 420 is illustrated in FIG. 7 sealing the second end 414 of the plug 410. The stopper 420 may be made from a radiolucent material such that one or more physical substances are contained within the open space 415, although radiation is allowed to escape through the stopper 420. The stopper 420 may also be made from or include material that substantially blocks the transmission of radiation. The stopper 420 may be coupled to the second end 414 by an interference fit, by threaded connection, by an adhesive, or by any effective mechanism. A similar stopper may be used in a first end where the first end is open, such as in the hole 217 of the first end 213 illustrated in FIGS. 3 and 5.

The plug 410 shown in FIG. 7 includes protrusions 419 along at least a portion of the plug 410. The protrusions 419 of some embodiments are for engaging with an interbody spinal implant or a vertebral body replacement implant, such as the vertebral body replacement implants 10, 110 illustrated in FIGS. 1 and 2 respectively. The protrusion 419 may serve to keep the plug 410 from sliding or otherwise translating relative to an interbody spinal implant or a vertebral body replacement implant. For example and without limitation, a portion of the vertebral body replacement implants 10, 110 adjacent to an opening in the implants may be captured between the flange 412 and one or more of the protrusions 419 to restrict the movement of the plug 410 relative to at least one of the vertebral body replacement implants 10, 110. The protrusions 419 may be made at least in part from a resilient material such that they may be compressed, deformed, or otherwise altered to be forced through an opening and then return to an original form to restrict movement of the plug 410.

The plug 510 is illustrated in FIGS. 1, 2, and 8, and may be coupled to an interbody implant or a vertebral body replacement implant by at least in part occupying a portion of an opening in an implant, such as the openings 12, 112 (FIGS. 1 and 2) in the vertebral body replacement implants 10, 110. The plug 510 shown includes a body extending between a first end 513 and a substantially opposite end 514. The plug 510 includes an open space 515 within the plug body. In other embodiments, for example where a radiation source is integrated into the material of a plug, the plug may be solid and not include an open space in the body. A plug designated a solid herein may be composed of more than one solid material and still be considered a solid. That is, designation as a solid does not necessarily mean that a plug is a homogeneous material. A connection mechanism of some embodiments may include an external hex, an internal hex, various flat portions, holes for receiving other components of a torque or other force delivering tool, or any other effective mechanism to connect with and manipulate the plug 510 from any end or side.

The plug 510 includes a first hole 517 from the open space 515 through the first end 513 and a second hole 518 from the open space 515 through the second end 514. In some embodiments, multiple holes may be provided from the open space 515 through each of the first end 513 and the second end 514. Either or both of the first end 513 and the second end 514 may be closed in some embodiments. This closure may be by a radiolucent material in some embodiments where it is desirable for radiation to emanate from an end, or the closure may be made with a material that substantially blocks the transmission of radiation. A stopper, such as the stopper 420 illustrated in FIG. 7, may be used to seal one or both of the first end 513 and the second end 514 of the plug 510.

The plug 510 shown in FIG. 8 includes protrusions 519, 520 along at least a portion of the plug 510. The protrusions 519, 520 of some embodiments are for engaging with an interbody spinal implant or a vertebral body replacement implant, such as the vertebral body replacement implants 10, 110 illustrated in FIGS. 1 and 2 respectively. The protrusion 519, 520 may serve to keep the plug 510 from sliding or otherwise translating relative to an interbody spinal implant or a vertebral body replacement implant. Fore example and without limitation, a portion of the vertebral body replacement implants 10, 110 adjacent to an opening in the implants may interfere with the protrusions 519, 520 collectively to restrict the movement of the plug 510 relative to at least one of the vertebral body replacement implants 10, 110. The protrusions 519, 520 may be made at least in part from a resilient material such that they may be compressed, deformed, or otherwise altered to be forced through an opening and then return to an original form to restrict movement of the plug 510.

Each of the plugs 210, 310, 410, 510, or other embodiments, may include any effective shape or configuration and be constructed from any biocompatible material or composite. Plug embodiments may be specifically configured to be press fit with or otherwise cooperatively engage particular interbody spinal implants or vertebral body replacement implants. In some embodiments, the plug 210, 310, 410, 510 is made at least in part from a material that substantially blocks the transmission of radiation. As used herein, the term “blocking the transmission of radiation” and similar terms mean that a material, composite, or component blocks the passage of therapeutically effective amounts of radiation from a radiation source. The blocking of radiation may not be complete such that there is no measurable amount of radiation allowed through a component. Non-limiting example materials that may block the transmission of radiation include cobalt chrome, titanium, stainless steel, tantalum, niobium, gold, lead, barium, bismuth, tin, and tungsten. A radiation blocking material may be applied to the inside or outside or be encapsulated within a component such as a plug so that only certain of the materials are in direct communication with tissues or fluids of a patient. A radiation blocking material may be applied to or integrated with a component by any effective mechanism, including but not limited to, chemically bonding, an intervening adhesive, welding, melting, press fitting, ion deposition, or mechanically locking.

The device for supplementing or replacing a spinal structure and therapeutically delivering radiation to tissue within or near the spinal structure may also include a radiation emitting device 1000, as illustrated in FIGS. 5-8, integrated with the plug and configured to deliver radiation. The radiation emitting device 1000 is shown within the respective open spaces 215, 315, 415, 515. In other embodiments, a radiation emitting device may be located at any effective location within, near, or on a plug. The direction of radiation transmission for some of the disclosed embodiments is through one or both of the first ends 213, 313, 413, 513 and the second ends 214, 314, 414, 514. Whether a particular end will allow radiation to be emitted may depend on whether the end is open or covered or has a stopper, and if covered or has a stopper, whether the material of the covering or stopper is radiolucent or is at least in part a material that blocks the transmission of radiation. The direction and pattern of radiation transmission may be further altered by the shape of the open space in the plug, the orientation of the open space, and the distance that the radiation emitting device 1000 is placed from the hole in open space through which the radiation is emitted. For example and without limitation, the radiation emitting device 1000 shown in FIG. 5 is place a relatively long distance from the hole 217 in the first end 213, and the open space 215 is relatively narrow. Therefore, radiation emitted from the first end 213 would be more narrowly focused than radiation from a plug that included the radiation emitting device 1000 place near the hole 217 in the first end 213.

The radiation emitting device 1000 may include any therapeutically effective radiation source. Suitable radiation sources for use in the radiation emitting device 1000 of some embodiments include both solids and liquids. By way of non-limiting example, the radiation source may be a radionuclide, such as I-125, I-131, Yb-169, Ir-192 or other radionuclides that emit photons, beta particles, gamma radiation, or other therapeutic energy or substances. The radioactive material may also be a fluid made from any solution of radionuclide(s), e.g., a solution of I-125 or I-131, or a radioactive mixture may be produced using a slurry of a suitable fluid containing small particles of solid radionuclides, such as Au-198, Y-90. Radionuclides may also be delivered in a gel. One radioactive material useful in some embodiments is Iotrex®, a nontoxic, water soluble, nonpyrogenic solution containing sodium 3-(125I)iodo-4-hydroxybenzenesulfonate (125I-HBS), available from Proxima Therapeutics, Inc. of Alpharetta, Ga. Radioactive micro spheres of the type available from the 3M Company of St. Paul, Minn., may also be used. A radioactive source may be preloaded into a plug at the time of manufacture, at some other time prior to a surgical procedure, or loaded after the plug has been implanted. By way of further non-limiting example, one or more solid radioactive micro spheres may be inserted through a catheter on a wire and into a plug.

Any of the interbody spinal implants or vertebral body replacement implants described above may be filled in whole or in part with an osteogenic material or therapeutic composition. Osteogenic materials include, without limitation, autograft, allograft, xenograft, demineralized bone, synthetic and natural bone graft substitutes, such as bioceramics and polymers, and osteoinductive factors. A separate carrier to hold materials within the device may also be used. These carriers may include collagen-based carriers, bioceramic materials, such as BIOGLASS®, hydroxyapatite and calcium phosphate compositions. The carrier material may be provided in the form of a sponge, a block, folded sheet, putty, paste, graft material or other suitable form. The osteogenic compositions may include an effective amount of a bone morphogenetic protein (BMP), transforming growth factor β1, insulin-like growth factor, platelet-derived growth factor, fibroblast growth factor, LIM mineralization protein (LMP), and combinations thereof or other therapeutic or infection resistant agents, separately or held within a suitable carrier material.

Embodiments of the invention may be applied to the lumbar spinal region, and embodiments may also be applied to the cervical or thoracic spine or between other skeletal structures.

Some embodiments may also include supplemental fixation devices in addition to or as part of the interbody spinal implants or vertebral body replacement implants disclosed herein for further supplement or replace spinal structures. For example, and without limitation, rod and screw fixation systems, anterior, posterior, or lateral plating systems, facet stabilization systems, spinal process stabilization systems, and any devices that supplement stabilization or replace spinal structures may be used as a part of or in combination with the interbody spinal implants or vertebral body replacement implants.

An embodiment of the invention is a method of irradiating cells near an interbody spinal implant or a vertebral body replacement implant. In some embodiments, a plug is provided that is configured to couple with the interbody spinal implant or the vertebral body replacement implant. For example and without limitation, a plug provided may include any of the plugs 210, 310, 410, 510 disclosed herein and variations. The plug may at least in part occupy some portion of an opening in the interbody spinal implant or the vertebral body replacement implant. Non-limiting example openings include the openings 12 and 112 illustrated in FIGS. 1 and 2 respectively. A radiation source, such as the radiation source 1000 (FIGS. 5-8) or other appended or integrated radiation source may also be provided.

Method embodiments may include coupling one or more of the plugs to the interbody spinal implant or the vertebral body replacement implant. This coupling may be accomplished by any act effective for the one or more plugs with a particular interbody spinal implant or vertebral body replacement implant. In the embodiments illustrated in FIGS. 1 and 2, the plug 210 may be placed simply by pushing the plug 210 into an opening 12, 112. The flange 212 (FIGS. 3 and 5) may serve as a stop to limit insertion depth. The plug 310, also illustrated in FIGS. 1 and 2, can be pushed into an opening in some embodiments where the threads 319 (FIGS. 4 and 6) are resilient or the plug 310 fits more loosely in an opening 12, 112. The plug 310 may alternatively or in combination be turned about its longitudinal axis to screw the plug 310 into an opening by use of the threads 319. The flange 312 may serve as a stop to limit insertion depth. Similarly, the plug 410 (FIG. 7) may be pushed into an opening, such as the openings 12, 112, and past the protrusions 419. The flange 412 may serve as a stop to limit insertion depth. Yet another example, the plug 510, illustrated in FIGS. 1, 2, and 8, may be placed from either end of the plug 510 into openings 12, 112 through the vertebral body replacement implants 10, 110. The plug 510 is an example of a plug that may extend fully across a lateral diameter of an interbody spinal implant or a vertebral body replacement implant.

Embodiments of a method for irradiating cells near an interbody spinal implant or a vertebral body replacement implant may also include inserting the interbody spinal implant or the vertebral body replacement implant into a patient. The method and direction of insertion may vary and may include any effective insertion technique.

Embodiments of a method for irradiating cells near an interbody spinal implant or a vertebral body replacement implant may also include inserting a plug, such as one or more of the plugs 210, 310, 410, 510 into a patient such that radiation is directed toward a therapeutically effective location near the interbody spinal implant or the vertebral body replacement implant. Therapeutically effective locations may include locations where a tumor or cancerous cells are present or suspected to be present, or areas from which a tumor or cancerous growth has been surgically removed. Therapeutically effective locations may also include areas where tissue growth is to be retarded, such as but not limited to, typical areas of scar tissue growth.

Depending on the configuration of the plug and integrated radiation source, radiation may be emitted from one or more directions from the plug. For example and without limitation, where a plug that emits radiation from its trailing end is inserted into an implant, the implant and inserted plug may be positioned to apply radiation to tissue at least adjacent to the point of insertion of the plug into the implant. Where a plug that emits radiation from its leading end is inserted into an implant, the implant and inserted plug may be positioned to apply radiation to tissue at least on the opposite side from the point of insertion of the plug into the implant. Plugs that emit radiation from both leading and trailing ends may be positioned to apply radiation to tissue at least on the opposite side from the point of insertion of the plug into the implant and adjacent to the point of insertion of the plug into the implant.

In some embodiments, one or more plugs may be coupled to an interbody spinal implant or a vertebral body replacement implant before inserting the interbody spinal implant or the vertebral body replacement implant into a patient. For example and without limitation, one or more of each of the plugs 210, 310, 410, 510 may be coupled to the vertebral body replacement implants 10, 110 before the vertebral body replacement implants are inserted into a patient. For such an act to be accomplished accurately, some embodiments may also include one or more of the placement of a trial, the use of markers, and radiographic or surgical navigation imaging or simulation. In some embodiments, one or more plugs may be coupled to the interbody spinal implant or the vertebral body replacement implant after inserting the interbody spinal implant or the vertebral body replacement implant into a patient. In some circumstances one or more plugs may be coupled to the interbody spinal implant or the vertebral body replacement implant before an implant is inserted into a patient, and then one or more additional plugs may be coupled to the implant once the implant is in a patient.

In some embodiments, an interbody spinal implant or the vertebral body replacement implant may be inserted into a patient along with a plug such that radiation is directed toward a therapeutically effective location near the interbody spinal implant or the vertebral body replacement implant by a single act. In some embodiments, an implant with a coupled plug may be inserted and then later further positioned to direct radiation toward a therapeutically effective location. For any of the disclosed method embodiments, additional plugs may be inserted into a patient to direct additional radiation toward one or more therapeutically effective locations near the interbody spinal implant or the vertebral body replacement implant, as needed.

An additional act of various method embodiments is to insert one or more radiation sources into one or more plugs. The one or more radiation sources may be inserted at any time during the treatment. For example and without limitation, all or a part of the radiation source may be inserted into one or more of the plugs prior to placement of the plugs into a patient or prior to placement of the implant into a patient. Alternatively or in addition, the radiation source or component parts of the radiation source may be inserted into the implant after it is in place in a patient or partially in place in a patient. The radiation source or components of the radiation source may be inserted one or more of pre-operatively, inter-operatively, and post-operatively. The radiation source may be a device capable of receiving radiation or components that emit radiation and may not at all times be able to emit radiation. That is, its designation as a “radiation source” does not mean that it, or one or more of its component parts, are at all times capable of emitting radiation.

Embodiments of the device for supplementing or replacing a spinal structure and therapeutically delivering radiation may be implanted from any surgical approach, including but not limited to, posterior, lateral, anterior, transpedicular, lateral extracavitary, in conjunction with a laminectomy, in conjunction with a costotransversectomy, or by any combination of these and other approaches.

Various method embodiments of the invention are described herein with reference to particular implants. However, in some circumstances, each disclosed method embodiment may be applicable to each of the implants, or to some other implant operable as disclosed with regard to the various method embodiments.

Terms such as anterior, posterior, lateral, side, leading, trailing, and the like have been used herein to note relative positions. However, such terms are not limited to specific coordinate orientations, but are used to describe relative positions referencing particular embodiments. Such terms are not generally limiting to the scope of the claims made herein.

While embodiments of the invention have been illustrated and described in detail in the disclosure, the disclosure is to be considered as illustrative and not restrictive in character. All changes and modifications that come within the spirit of the invention are to be considered within the scope of the disclosure.

What is claimed is:

1. A device for supplementing or replacing a spinal structure and therapeutically delivering radiation to tissue within or near the spinal structure comprising:
an interbody spinal implant or a vertebral body replacement implant configured to be placed between a first vertebra and a second vertebra to supplement or replace at least a portion of the spinal structure, wherein the interbody spinal implant or vertebral body replacement implant includes at least one opening;
a plug coupled to the interbody spinal implant or vertebral body replacement implant by at least in part occupying some portion of the at least one opening; and
a radiation source integrated with the plug and configured to deliver radiation.

2. The device of claim 1 wherein the interbody spinal implant or the vertebral body include a mesh material wherein one or more of the openings in the mesh is a generally polygonal shape.

3. The device of claim 2 wherein the generally polygonal shape is generally a triangular shape.

4. The device of claim 1 wherein the plug includes a flange at the first end.

5. The device of claim 4 wherein the flange includes a connection mechanism for applying torque to the plug.

6. The device of claim 1 wherein the plug comprises a body having a first end and a substantially opposite second end and having at least some open space within the body.

7. The device of claim 6 wherein the plug is made at least in part from a material that substantially blocks the transmission of radiation.

8. The device of claim 6 wherein the body includes a hole from the open space through the first end and the body includes a closed second end.

9. The device of claim 6 wherein the body includes a hole from the open space through the second end and the body includes a closed first end.

10. The device of claim 6 wherein the body includes a hole from the open space through the first end and the body includes a hole from the open space through the second end.

11. The device of claim 6 wherein a hole from the open space through the first end is sealed with a radiolucent material.

12. The device of claim 6 wherein a hole from the open space through the second end is sealed with a radiolucent material.

13. The device of claim 1 wherein the plug includes one or more threads along at least a portion of the plug for engaging with the interbody spinal implant or the vertebral body replacement implant.

14. The device of claim 1 wherein the plug includes one or more protrusions along at least a portion of the plug for engaging with the interbody spinal implant or the vertebral body replacement implant.

15. A plug configured to couple with an interbody spinal implant or a vertebral body replacement implant by at least in part occupying some portion of an opening in the interbody spinal implant or the vertebral body replacement implant, wherein the plug comprises a body having a first end and a substantially opposite second end and having at least some open space within the body; and wherein the plug is made at least in part from a material that substantially blocks the transmission of radiation to direct radiation from only one or both of the first end and the second end of the plug.

16. The device of claim 15 wherein the plug includes a flange at the first end.

17. The device of claim 16 wherein the flange includes a connection mechanism for applying torque to the plug.

18. The device of claim 15 wherein the body includes a hole from the open space through the first end and the body includes a closed second end.

19. The device of claim 15 wherein the body includes a hole from the open space through the second end and the body includes a closed first end.

20. The device of claim 15 wherein the body includes a hole from the open space through the first end and the body includes a hole from the open space through the second end.

21. The device of claim 15 wherein a hole from the open space through the first end is sealed with a radiolucent material.

22. The device of claim 15 wherein a hole from the open space through the second end is sealed with a radiolucent material.

23. The device of claim 15 wherein the plug includes one or more threads along at least a portion of the plug for engaging with the interbody spinal implant or the vertebral body replacement implant.

24. The device of claim 15 wherein the plug includes one or more protrusions along at least a portion of the plug for engaging with the interbody spinal implant or the vertebral body replacement implant.

25. A method of irradiating cells near an interbody spinal implant or a vertebral body replacement implant comprising:
providing a plug configured to couple with the interbody spinal implant or the vertebral body replacement implant by at least in part occupying some portion of an opening in the interbody spinal implant or the vertebral body replacement implant, wherein the plug includes a radiation source;
coupling the plug to the interbody spinal implant or the vertebral body replacement implant;
inserting the interbody spinal implant or the vertebral body replacement implant into a patient; and
inserting the plug into a patient such that radiation is directed toward a therapeutically effective location near the interbody spinal implant or the vertebral body replacement implant.

26. The method of claim 25 wherein the plug is coupled to the interbody spinal implant or the vertebral body replacement implant before inserting the interbody spinal implant or the vertebral body replacement implant into a patient.

27. The method of claim 25 wherein the plug is coupled to the interbody spinal implant or the vertebral body replacement implant after inserting the interbody spinal implant or the vertebral body replacement implant into a patient.

28. The method of claim 25 wherein the act of inserting the interbody spinal implant or the vertebral body replacement implant into a patient and the act of inserting the plug into a patient such that radiation is directed toward a therapeutically effective location near the interbody spinal implant or the vertebral body replacement implant are accomplished by the same act.

29. The method of claim 25, further comprising inserting one or more additional plugs into a patient to direct radiation toward one or more therapeutically effective locations near the interbody spinal implant or the vertebral body replacement implant.
